(19) United States
(12) Patent Application Publication
(10) Pub. No.: US 2022/0003182 A1
(43) Pub. Date: Jan. 6, 2022

(54) METHOD AND SYSTEM FOR DIAGNOSING MISFIRE OF ENGINE

(71) Applicants: HYUNDAI MOTOR COMPANY, Seoul (KR); KIA MOTORS CORPORATION, Seoul (KR)

(72) Inventors: Tae Kwang Eom, Hwaseong-si (KR); Jeonghun Lee, Seongnam-si (KR); Hyeseung Lee, Incheon (KR); Jeong Hwan Paeng, Seoul (KR); Jun Wook Jeong, Incheon (KR); Kyu Sup Kim, Seoul (KR)

(21) Appl. No.: 17/063,381

(22) Filed: Oct. 5, 2020

(30) Foreign Application Priority Data
Jul. 3, 2020 (KR) 10-2020-0081911

(51) Int. Cl. F02D 41/22; G01N 33/00; G01K 13/02; G01L 5/00; G01M 15/05; G07C 5/08

(52) U.S. Cl. CPC F02D 41/22; G01N 33/0036; G01K 13/02; G01L 5/00; G01M 15/05; F02D 2200/0406; G01K 2013/024; G01K 2205/02; G01K 2205/04; F02D 2200/1002; F02D 2041/224; G07C 5/085

(57) ABSTRACT

A system for diagnosing a misfire of an engine includes a sensing unit including at least one sensor for detecting at least one detection value associated with an operation of the engine, and an electronic control unit configured to determine whether a misfire of the engine due to exhaust valve leakage has occurred based on the detection values from the sensing unit, and perform an operation corresponding to the misfire due to exhaust valve leakage when the misfire due to exhaust valve leakage has occurred, wherein the electronic control unit a misfire code for exhaust valve leakage in a memory when the misfire due to exhaust valve leakage has occurred.

### CROSS-REFERENCE TO RELATED APPLICATION

This application claims priority to and the benefit of Korean Patent Application No. 10-2020-0081911 filed in the Korean Intellectual Property Office on Jul. 3, 2020, the entire contents of which are incorporated herein by reference.

### BACKGROUND

### (a) Field

The present disclosure relates to methods and systems for diagnosing a misfire of an engine.

### (b) Description of the Related Art

Generally, an engine is a device that generates power by combusting air and fuel in a cylinder.

In order for the air/fuel mixture to normally combust as expected in the cylinder, various variables such as the ratio of air and fuel, fuel injection timing, and ignition timing in the case of a gasoline engine must be operated as designed. Due to various factors, the air/fuel mixture in the cylinder may not be sufficiently (i.e., normally) combusted; such an instance is called a misfire.

When a misfire occurs, the fuel does not react sufficiently with air and may be exhausted unburned. For example, hydrocarbon (HC) may be exhausted in a large amount. Since unburned gas causes pollution, a misfire should be diagnosed as an on-board diagnostic (OBD) item. The unburned gas may cause excessive oxidation reactions in purifying devices such as catalytic converters, and damage components such as catalytic converters in an exhaust system of a vehicle.

A method and system capable of monitoring various misfires and analyzing or determining the cause thereof may be desired to reduce environmental problems such as pollution and improve durability of a vehicle purifying device.

The above information disclosed in this Background section is only for enhancement of understanding of the background of the disclosure.

### SUMMARY OF THE DISCLOSURE

An exemplary system for diagnosing a misfire of an engine includes a sensing unit including at least one sensor for detecting at least one detection value associated with an operation of the engine, and an electronic control unit configured to determine whether a misfire of the engine due to exhaust valve leakage has occurred based on the detection values from the sensing unit, and perform an operation corresponding to the misfire due to exhaust valve leakage when the misfire due to exhaust valve leakage has occurred. The electronic control unit may store a misfire code for exhaust valve leakage in a memory when the misfire due to exhaust valve leakage has occurred.

The electronic control unit may be configured to detect an output torque drop of the engine, control the engine by an optimal air/fuel ratio when the output torque drop of the engine is detected, count an output torque drop of the engine while controlling the engine by the optimal air/fuel ratio, and determine whether the misfire due to exhaust valve leakage has occurred when the output torque drop count is above a predetermined number.

The misfire code for exhaust valve leakage may include information on a misfire occurrence driving point where the misfire due to exhaust valve leakage has occurred.

When the misfire due to exhaust valve leakage has occurred, the electronic control unit may control the engine at a driving point avoiding the misfire occurrence driving point.

The electronic control unit may be configured to determine whether a code corresponding to a misfire due to an injection fail and/or an ignition fail is already stored in the memory when the output torque drop count is above a predetermined number, additionally store a further code indicating that a misfire has additionally occurred when the code corresponding to a misfire due to an injection fail and/or an ignition fail is already stored in the memory, and perform the determining of whether the misfire due to exhaust valve leakage has occurred only when the code corresponding to a misfire due to an injection fail and/or an ignition fail is not stored in the memory.

The sensing unit may include a MAF sensor for detecting an intake air amount supplied to the engine, an upstream oxygen sensor for detecting upstream oxygen concentration of a catalytic converter of the engine, a MAP sensor for detecting an intake manifold pressure of the engine, an exhaust temperature sensor for detecting an exhaust temperature of the engine, and a manifold temperature sensor for detecting an intake manifold temperature the engine.

The electronic control unit may determine whether the misfire due to exhaust valve leakage has occurred, based on a combination of at least one of (a first condition) whether the intake air amount detected by the MAF sensor has decreased compared to a normal combustion, (a second condition) whether the upstream oxygen concentration detected by the upstream oxygen sensor has increased compared to the normal combustion, (a third condition) the intake manifold pressure detected by the MAP sensor has increased compared to the normal combustion, (a fourth condition) whether the exhaust temperature detected by the exhaust temperature sensor has increased compared to the normal combustion, and (a fifth condition) whether the intake manifold temperature detected by the manifold temperature sensor has increased compared to the normal combustion.

In this case, the electronic control unit may determine that the misfire due to exhaust valve leakage has occurred when the first condition, the second condition, the third condition, and the fifth condition are all satisfied.

Alternatively, the electronic control unit may determine that the misfire due to exhaust valve leakage has occurred when the first condition, the second condition, the third condition, the fourth condition, and the fifth condition are all satisfied.

In an exemplary variation, the sensing unit may include a MAF sensor for detecting an intake air amount supplied to the engine, an upstream oxygen sensor for detecting upstream oxygen concentration of a catalytic converter of the engine, a MAP sensor for detecting an intake manifold pressure of the engine, and an exhaust temperature sensor for detecting an exhaust temperature of the engine. The electronic control unit may determine whether the misfire due to exhaust valve leakage has occurred based on a combination of at least one of (a first condition) whether the intake air amount detected by the MAF sensor has decreased compared to a normal combustion, (a second condition) whether the upstream oxygen concentration detected by the upstream oxygen sensor has increased compared to the normal combustion, (a third condition) the intake manifold pressure detected by the MAP sensor has increased compared to the normal combustion, and (a fourth condition) whether the exhaust temperature detected by the exhaust temperature sensor has increased compared to the normal combustion.

In this case, the electronic control unit may determine that the misfire due to exhaust valve leakage has occurred when the first condition, the second condition, and the third condition are all satisfied.

Alternatively, the electronic control unit may determine that the misfire due to exhaust valve leakage has occurred when the first condition, the second condition, the third condition, and the fourth condition are all satisfied.

An exemplary method for diagnosing a misfire of an engine includes receiving a detection value from a sensing unit including at least one sensor for detecting at least one detection value associated with an operation of the engine, detecting an output torque drop of the engine, controlling the engine by an optimal air./fuel ratio when the output torque drop of the engine is detected, counting an output torque drop of the engine while controlling the engine by the optimal air/fuel ratio, determining whether a misfire due to exhaust valve leakage has occurred when the output torque drop count is above a predetermined number, and storing a misfire code in a memory when the misfire due to exhaust valve leakage has occurred.

The storing of the misfire code into the memory may include storing information on a misfire occurrence driving point where the misfire due to exhaust valve leakage has occurred in the memory.

An exemplary method for diagnosing a misfire of an engine may further include controlling the engine at a driving point avoiding the misfire occurrence driving point when the misfire due to exhaust valve leakage has occurred.

An exemplary method for diagnosing a misfire of an engine may further include determining whether a code corresponding to a misfire due to an injection fail and/or an ignition fail is already stored in the memory when the output torque drop count is above a predetermined number, and additionally storing a further code indicating that a misfire has additionally occurred when the code corresponding to a misfire due to an injection fail and/or an ignition fail is already stored in the memory. Here, the determining of whether the misfire due to exhaust valve leakage has occurred may be performed when the code corresponding to a misfire due to an injection fail and/or an ignition fail is not stored in the memory.

Whether the misfire due to exhaust valve leakage has occurred may be determined based on a combination of at least one of (a first condition) whether an intake air amount supplied to the engine has decreased compared to a normal combustion, (a second condition) whether an upstream oxygen concentration of a catalytic converter of the engine has increased compared to the normal combustion, (a third condition) whether an intake manifold pressure of the engine has increased compared to the normal combustion, (a fourth condition) whether an exhaust temperature of the engine has increased compared to the normal combustion, and (a fifth condition) whether an intake manifold temperature of the engine has increased compared to the normal combustion,

In this case, it may be determined that the misfire due to exhaust valve leakage has occurred when the first condition, the second condition, the third condition, and the fifth condition are all satisfied.

Alternatively, it may be determined that the misfire due to exhaust valve leakage has occurred when the first condition, the second condition, the third condition, the fourth condition, and the fifth condition are all satisfied.

Whether the misfire due to exhaust valve leakage has occurred may be determined based on a combination of at least one of (a first condition) whether an intake air amount supplied to the engine has decreased compared to a normal combustion, (a second condition) whether an upstream oxygen concentration of a catalytic converter of the engine has increased compared to the normal combustion, (a third condition) whether an intake manifold pressure of the engine has increased compared to the normal combustion, and (a fourth condition) whether an exhaust temperature of the engine has increased compared to the normal combustion.

In this case, it may be determined that the misfire due to exhaust valve leakage has occurred when the first condition, the second condition, and the third condition are all satisfied.

According to a method and system for diagnosing a misfire of an engine according to an exemplary embodiment, a misfire due to exhaust valve leakage may be diagnosed. Therefore, the cause of misfires that may be diagnosed is diversified. Furthermore, even if the misfire due to exhaust valve leakage occurs, the engine may be stably controlled.

Other effects that may be obtained or are predicted by an exemplary embodiment will be explicitly or implicitly described in a detailed description of the present disclosure. That is, various effects that are predicted according to an exemplary embodiment will be described in the following detailed description.

### BRIEF DESCRIPTION OF THE DRAWINGS

FIG. 1 is a schematic diagram illustrating an engine applied with a system for diagnosing a misfire of an engine according to an exemplary embodiment,

FIG. 2 is a block diagram illustrating a system for diagnosing a misfire of an engine according to an exemplary embodiment.

FIG. 3 is a table summarizing changes in exemplary detection values measured by sensors when a misfire occurs in an engine.

FIG. 4A to FIG. 4F are diagrams showing exemplary values of each sensor detected in normal combustion and exemplary misfire situations.

FIG. 5 is a flowchart illustrating a method for diagnosing a misfire of an engine according to an exemplary embodiment.

### DETAILED DESCRIPTION

The present disclosure will be described more fully hereinafter with reference to the accompanying drawings, in which exemplary embodiments of the disclosure are shown. As those skilled in the art would realize, the described embodiments may be modified in various different ways, all without departing from the spirit or scope of the present disclosure.

Further, in exemplary embodiments, since like reference numerals designate like elements having the same configuration, a first exemplary embodiment is representatively described, and in other exemplary embodiments, only different configurations from the first exemplary embodiment will be described.

In order to clarify the present disclosure, parts that are not related to the description will be omitted, and the same elements or equivalents are referred to with the same reference numerals throughout the specification.

In addition, unless explicitly described to the contrary, the word “comprise” and variations such as “comprises” or “comprising”, will be understood to imply the inclusion of stated elements but not the exclusion of any other elements.

Throughout this specification and the claims that follow, when it is described that an element is “coupled” to another element, the element may be “directly coupled” to the other element or “electrically coupled” to the other element through a third element. In addition, unless explicitly described to the contrary, the word “comprise”; and variations such as “comprises” or “comprising”, will be understood to imply the inclusion of stated elements but not the exclusion of any other elements.

In the following description, dividing names of components into first, second, and the like is to divide the names because the names of the components are the same as each other, and an order thereof is not particularly limited.

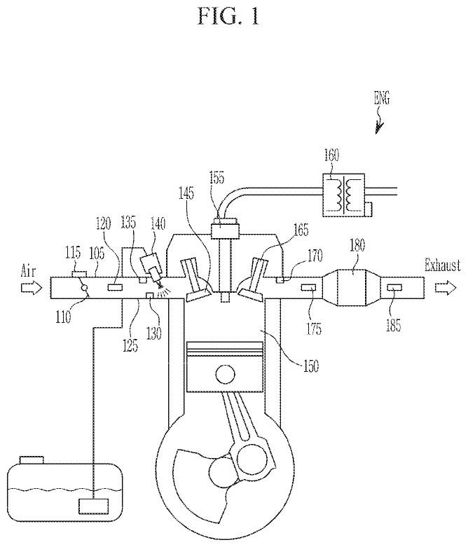

FIG. 1 is a schematic diagram illustrating an engine applied with a system for diagnosing a misfire of an engine according to an exemplary embodiment.

An exemplary embodiment attempts to provide a method and system that are capable of diagnosing a misfire of an engine, including a misfire due to exhaust valve leakage, based on detection values by sensors provided in an engine.

If the cause of various misfires may be determined more accurately, it is possible to more accurately specify the parts required for maintenance.

As shown in FIG. 1, an engine ENG that is the subject of misfire diagnosis according to an exemplary embodiment, receives intake air into a cylinder 150 and injects a corresponding amount of fuel by an injector 140 into the cylinder 150, thereby generating power by combusting the fuel.

The engine ENG is equipped with a throttle valve 110 for controlling an intake air amount, and the throttle valve 110 is equipped with a throttle position sensor 115 for detecting a throttle valve opening. When the throttle valve 110 is open, the air may be drawn into an intake route 105. An intake air amount sensor (e.g., a mass air flow (MAF) sensor; hereinafter, referred to as a “MAF sensor”) 120 is disposed on the intake route 105 to detect the intake air amount supplied into the engine ENG.

The engine ENG is equipped with an intake valve 145 to receive the intake air into the cylinder 150, and equipped with an exhaust valve 165 to exhaust combustion gas out of the cylinder 150.

In the following description, it may be understood that the intake manifold of which a temperature and a pressure are detected includes a surge tank.

According to the operation of the engine ENG, for example, according to the operation of the throttle valve 110 and the intake valve 145, a pressure within the intake manifold 125 varies, and a manifold pressure sensor (e.g., a manifold absolute pressure (MAP) sensor; hereinafter, referred to as a “MAP sensor”) 130 to detect the pressure within the intake manifold 125 is disposed at the intake manifold 125.

Additionally, a manifold temperature sensor 135 for detecting a temperature in the intake manifold 125 may be disposed to the intake manifold 125.

For normal operation of the engine ENG, it is necessary to detect the intake air temperature. As an example, a temperature sensor (not shown) may be employed in the MAF sensor 120 to detect intake air temperature. As another example, when the manifold temperature sensor 135 is provided, the intake air temperature may be detected by the manifold temperature sensor 135.

The engine ENG calculates the intake air amount based on an intake air amount detected by the MAF sensor 120, an intake manifold pressure detected by the MAP sensor 130, and intake air temperature detected by the MAF sensor 120 and/or the manifold temperature sensor 135. Then, the engine ENG forms an air/fuel mixture by injecting fuel of an amount corresponding to the calculated intake air amount by the injector 140, and combusts the air/fuel mixture in the cylinder 150.

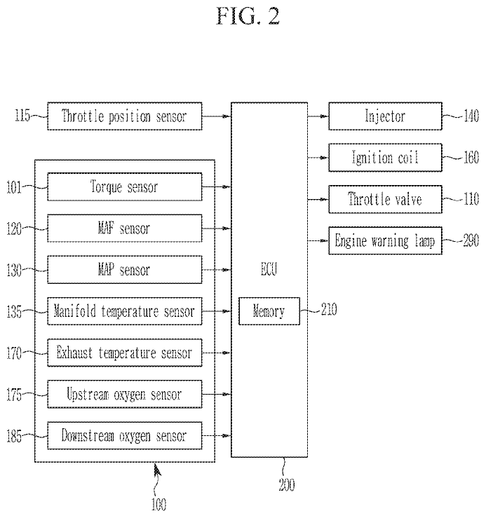

The injector 140 receives fuel through a fuel line from the fuel tank, and injects an amount of fuel specified by the control of an electronic control unit (ECU) 200 (as shown in FIG. 2) at a specified time (injection timing).

FIG. 1 illustrates the injector 140 disposed upstream of the intake valve 145, but the embodiments of the present disclosure are not necessarily limited thereto. A misfire diagnosis according to an exemplary embodiment may be applied to an engine that directly injects fuel into the cylinder 150, such as a gasoline direct injection (GDI) engine.

A spark plug 155 driven by an ignition coil 160 is disposed on one side of the cylinder 150 (e.g., at a top of the cylinder) to trigger the combustion of the air/fuel mixture by igniting the air/fuel mixture in the cylinder 150.

Combustion gas combusted in the cylinder 150 is exhausted to the outside of the cylinder 150 by the operation of the exhaust valve 165, and forms exhaust gas. As the exhaust gas passes through the catalytic converter 180, complete combustion of the unburned portion is promoted and noxious gases are removed.

In the exhaust system of the engine ENG, an exhaust temperature sensor 170 that detects the temperature of the exhaust gas exhausted from the exhaust stroke is disposed. An upstream oxygen sensor 175 is disposed upstream of the catalytic converter 180 of the engine ENG, to detect an oxygen concentration of the exhaust gas flowing through the upstream of the catalytic converter 180 and to generate a corresponding signal. A downstream oxygen sensor 185 may be additionally disposed downstream of the catalytic converter 180, to detect the oxygen concentration of exhaust gas flowing through downstream of the catalytic converter 180 and to generate a corresponding signal.

The engine ENG includes an electronic control unit (ECU) 200 that controls overall operation of the engine ENG. The ECU 200 controls the injector 140 and the ignition coil 160 based on detection values of various sensors disposed upstream and downstream of the cylinder 150. As needed, the ECU 200 may additionally control the throttle valve 110 (e.g., in a throttle system called a throttle-by-wire).

The ECU 200 monitors the operation of the engine ENG based on the detection values of various sensors disposed in the upstream and the downstream of the cylinder 150. In monitoring the operation of the engine ENG, the ECU 200 may monitor a misfire of the engine ENG, and accordingly may control the operation of the engine ENG differently.

FIG. 2 is a block diagram illustrating a system for diagnosing a misfire of an engine according to an exemplary embodiment.

As shown in FIG. 2, a system for diagnosing a misfire of an engine according to an exemplary embodiment includes a sensing unit 100 for measuring detection values used for diagnosing misfire of the engine ENG.

The sensing unit 100 includes a torque sensor 101 for detecting the output torque of the engine ENG, the MAF sensor 120 for detecting the intake air amount supplied to the engine ENG, the MAP sensor 130 for detecting the pressure in the intake manifold, and the exhaust temperature sensor 170 for detecting the exhaust gas temperature exhausted in the exhaust stroke of the engine ENG. The MAF sensor 120 may additionally detect an intake air temperature in addition to the intake air amount supplied to the engine ENG.

In addition, the sensing unit 100 further includes the upstream oxygen sensor 175 that is disposed in the upstream of the catalytic converter 180 of the engine ENG, and detects oxygen concentration of the exhaust gas flowing through the upstream of the catalytic converter, thereby generating a corresponding signal.

Optionally, the sensing unit 100 may further include the manifold temperature sensor 135 for detecting a temperature in the intake manifold 125. In addition, the sensing unit 100 may further include the downstream oxygen sensor 185 that is disposed downstream of the catalytic converter 180, and detects oxygen concentration of the exhaust gas flowing through the downstream of the catalytic converter 180, thereby generating a corresponding signal.

A system for diagnosing a misfire of an engine according to an exemplary embodiment further includes the ECU 200, and the ECU 200 determines a misfire of the engine ENG based on the detection value from the sensing unit 100, and performs an operation corresponding thereto.

That is, a system for diagnosing a misfire of an engine according to an exemplary embodiment is intended to perform misfire determination and corresponding operation by the ECU 200 that controls the overall operation of the engine ENG. However, the present disclosure is not limited thereto. It is also possible to perform the misfire determination of the engine according to an exemplary embodiment by a separate electronic control unit from the ECU 200 that controls the overall operation of the engine.

In an example embodiment, the torque sensor 101 may be implemented as a physical sensor that measures an actual torque of the engine ENG. In another example, the ECU 200 may calculate the output torque of the engine ENG from detection values obtained from various sensors of the engine ENG. In this case, at least one sensor that provides detection values based on the calculation of the output torque by the ECU 200 may be referred to as the torque sensor 101.

In a system for diagnosing a misfire of an engine according to an exemplary embodiment, the ECU 200 may control the operation of the engine ENG by controlling the injector 140 and the ignition coil 160, or by additionally controlling the throttle valve 110.

A memory 210 is installed in the ECU 200, and the ECU 200 stores a misfire code corresponding to the memory 210 when a misfire of the engine ENG is determined based on a detection value from the sensing unit 100.

In addition, the ECU 200 may warn the driver by lighting an engine warning lamp 290 when the misfire of the engine ENG is determined.

The ECU 200 may be implemented with at least one microprocessor operable by a predetermined program, and the predetermined program may include a set of instruction for performing each step included in a method for diagnosing a misfire of an engine according to an exemplary embodiment described below.

The misfire code may include whether a misfire has occurred, the cause of the misfire, and other information about the misfire as needed,

In addition to the misfire due to an injection fail and/or an ignition fail, a system for diagnosing a misfire of an engine according to an exemplary embodiment may also determine a misfire due to exhaust valve leakage. As for the determination of the misfire due to an injection fail and/or an ignition fail, there are methods known to those skilled in the art, and thus additional description is omitted.

Exhaust valve leakage means that exhaust gas leaks backward into the cylinder 150 from the exhaust port of the engine ENG because the exhaust valve is not completely closed.

As the operation of the engine ENG accumulates, the operation of the exhaust valve accumulates, and accordingly, when the valve is degraded due to one-sided wear, etc., a state in which the valve is not completely closed may occur. In addition, a leak in the valve may be caused when soot is accumulated on the valve seat surface. In addition, when the elastic force of a valve spring of the exhaust valve is deteriorated, closing operation of the exhaust valve slows down, and the period during which the exhaust valve is open is unintentionally prolonged, such that exhaust gas may flow back into the cylinder.

Hereinafter, the principle by which a misfire due to exhaust valve leakage may be determined is described in detail.

If leakage occurs due to unintended opening in the process of closing the exhaust valve, exhaust gas may be flow back into the cylinder, which acts as an effect that exhaust gas is not sufficiently exhausted and remains in the cylinder.

Therefore, this will affect the subsequent intake stroke to decrease the intake air amount.

When the intake air amount is reduced in the intake stroke compared to an intended amount by the exhaust gas remaining in the cylinder, the flow rate of the intake is lowered, so the temperature of the intake system (e.g., the temperature of the intake manifold) becomes higher than intended, and the pressure in the intake manifold (e.g., a surge tank) rises above the intended level.

In an instance of misfire due to an injection fail or an ignition fail, the exhaust temperature decreases rapidly. In contrast, although the misfire due to exhaust valve leakage may occur by the residual gas caused by the backward flow of the exhaust gas, the exhaust temperature (i.e., the temperature of the exhaust gas) is formed higher in comparison with the misfire due to an injection fail or an ignition fail,

In the instance of a misfire due to exhaust valve leakage compared to the general misfire due to an injection fail and/or an ignition fail, the amount of intake air is greatly reduced and fuel is injected accordingly. Therefore, the oxygen concentration detected by the upstream oxygen sensor 175 becomes a slightly increased level as compared with the normal combustion, and becomes much lower (a voltage of an oxygen sensor becomes much higher) than the oxygen concentration of the upstream oxygen sensor 175 at misfire due to an ignition fail and/or an injection fail.

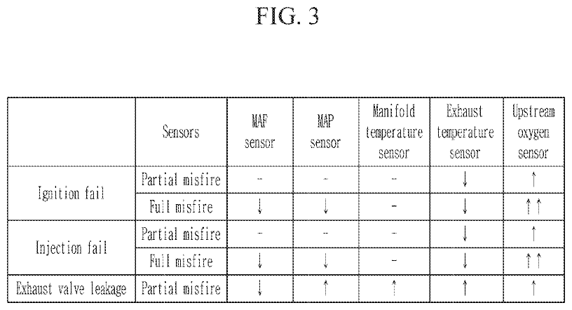

FIG. 3 is a table summarizing changes in exemplary detection values measured by sensors when a misfire occurs in an engine. FIG. 3 is an example of a partial misfire and/or a full misfire at a high speed operation, for example, 5,000 rpm or more, of a turbo GDI engine. It may be understood that specific values may vary depending on the specifications of the specific engine and the operating state at the time of the misfire.

In FIG. 3, the mark “−” indicates a negligible change compared to the normal combustion. The mark “↓” indicates a small decrease (i.e., by more than a first predetermined ratio) compared to the normal combustion. The mark “↑” indicates a small increase (i.e., by more than a first predetermined ratio) compared to the normal combustion. The mark “↑↑” indicates a large increase (i.e., by more than a second predetermined ratio larger than the first predetermined ratio) compared to the normal combustion.

The first predetermined ratio and the second predetermined ratio may be set as different values depending on sensors. For example, a 2% change in the detection value may be regarded as the same for the MAF sensor 120, but may be regarded as being more than the first predetermined ratio for the manifold temperature sensor 135. The first predetermined ratio and the second predetermined ratio for the detection value of each sensor may be experimentally set according to the intention of the designer for a specific engine.

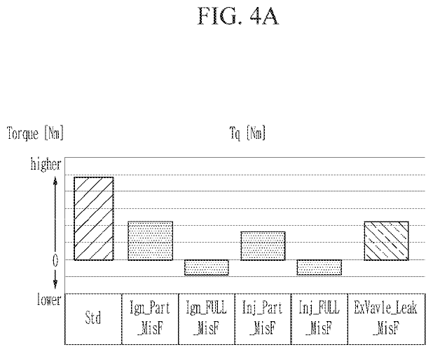

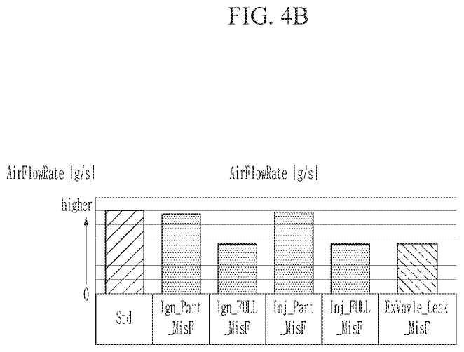

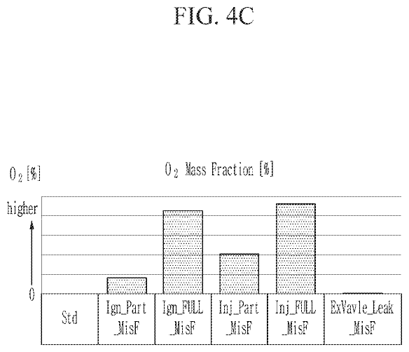

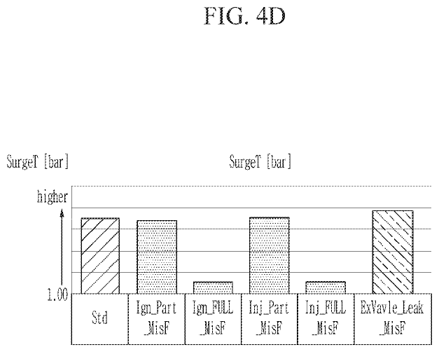

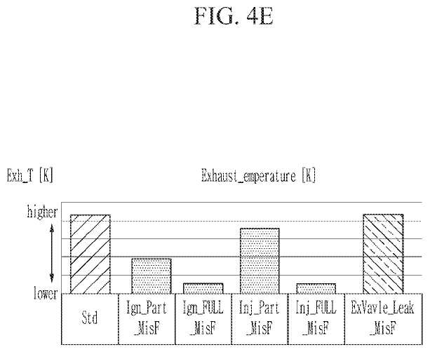

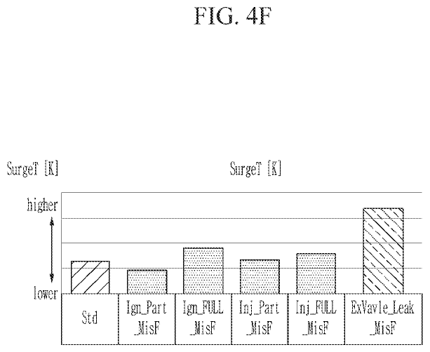

FIG. 4A to FIG. 4F are diagrams showing exemplary values of each sensor detected in normal combustion and exemplary misfire situations. FIG. 4A to FIG. 4F may be obtained at a same or a similar condition as for FIG. 3.

As mentioned above, the specific detection values generated by sensors at the engine misfire depend on the specific engine specifications and the operating state at the time of misfire. Therefore, FIG. 4A to FIG. 4F show qualitative character of how the detected values of sensors change depending on the cause of misfire, rather than showing specific values.

In FIG. 4A to FIG. 4F, “Std” indicates a normal combustion state, “Ign_Part_MisF” indicates a partial misfire due to an ignition fail, “Ign_Full_MisF” indicates a full misfire due to an ignition fail, “Inj_Part_MisF” indicates a partial misfire due to an injection fail, “Inj_Full_MisF” indicates a full misfire due to an injection fail, and “ExValve_Leak_MisF” indicates a misfire due to exhaust valve leakage.

FIG. 4A exemplifies the output torque of the engine ENG in a normal combustion and exemplary misfire situations.

FIG. 4B exemplifies the intake air amount detected by the MAF sensor 120 in a normal combustion and exemplary misfire situations.

FIG, 40 exemplifies an oxygen concentration (%) detected by the upstream oxygen sensor 175 in a normal combustion and exemplary misfire situations.

FIG. 4A exemplifies the intake manifold pressure detected by the MAP sensor 130 in a normal combustion and exemplary misfire situations.

FIG. 4E exemplifies the exhaust temperature detected by the exhaust temperature sensor 170 in a normal combustion and exemplary misfire situations.

FIG. 4F exemplifies the intake manifold temperature (e.g., surge tank temperature) detected by the manifold temperature sensor 135 in a normal combustion and exemplary misfire situations.

Referring to FIG. 3 and FIG. 4A to FIG. 4F, causes of misfires due to an ignition fail, an injection fail, and exhaust valve leakage may be understood, and various configurations for determining misfire based on the detection value from the sensing unit 100 may be understood. Hereinafter, the misfire due to exhaust valve leakage is more focused in the description.

Referring to FIG. 4A, when a misfire occurs, the output torque of the engine ENG is deteriorated. Therefore, as a basic process for misfire determination, the condition that the output torque is deteriorated may be included.

Referring to FIG. 4B, the detection value (i.e., the intake air amount) of the MAF sensor 120 does not show a substantial difference in a partial misfire due to an ignition fail and an injection fail in comparison with the normal combustion, and slightly decreases in a misfire due to exhaust valve leakage in comparison with the normal combustion, similarly to a partial misfire due to an ignition fail and an injection fail.

Referring to FIG. 4C, the detection value (i.e., the oxygen concentration) of the upstream oxygen sensor 175 slightly increases in a partial misfire due to an ignition fail and an injection fail in comparison with the normal combustion. The detection value (i.e., the oxygen concentration) of the upstream oxygen sensor 175 much increases in a full misfire due to an ignition fail and an injection fail in comparison with the normal combustion. The detection value (i.e., the oxygen concentration) of the upstream oxygen sensor 175 slightly increases in a misfire due to exhaust valve leakage in comparison with the normal combustion.

Referring to FIG. 4D, the detection value (i.e., the intake manifold pressure) of the MAP sensor 130 does not show a substantial difference in a partial misfire due to an ignition fail and an injection fail in comparison with the normal combustion, and slightly decreases in a full misfire due to an ignition fail and an injection fail in comparison with the normal combustion. Meanwhile, in a misfire due to exhaust valve leakage, the intake manifold pressure slightly increases in comparison with the normal combustion.

Referring to FIG. 4E, the detection value (i.e., the exhaust temperature) of the exhaust temperature sensor 170 slightly decreases in ignition fail and injection fail in comparison with the normal combustion, and slightly increases in a misfire due to exhaust valve leakage in comparison with the normal combustion.

Referring to FIG. 4F, the detection value (i.e., the intake manifold temperature) of the manifold temperature sensor 135 does not show a substantial difference in a misfire due to an ignition fail and an injection fail in comparison with the normal combustion, and slightly increases in a misfire due to exhaust valve leakage in comparison with the normal combustion.

Referring to these phenomena, based on the detection values detected from the sensing unit 100, it is possible to determine a misfire due to exhaust valve leakage, which cannot be determined while determining a misfire due to an ignition fail and an injection fail, which is described in detail below.

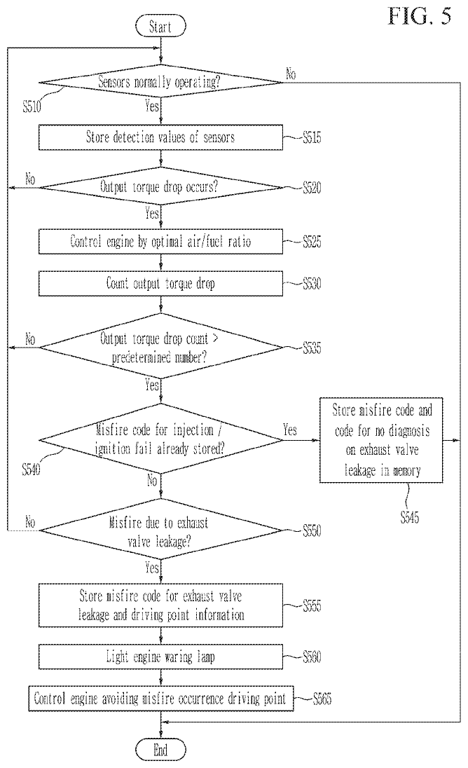

FIG. 5 is a flowchart illustrating a method for diagnosing a misfire of an engine according to an exemplary embodiment.

Firstly at step S510, the ECU 200 receives a detection value from each sensor in the sensing unit 100, and determines whether each sensor is normally operating. Whether a sensor is normally operating or not may be determined in various methods, for example, based on whether an output value of the sensor is within a predetermined normal range.

Subsequently at step S515, The ECU 200 stores the detection values of the sensors in the sensing unit 100 in the memory 210. At this step S515, output values of the sensors in the normal combustion may be stored, and may be used for determining whether a misfire occurs.

For example, the intake air amount may be stored with respect to the MAF sensor 120, the intake manifold pressure may be stored with respect to the MAP sensor 130, the intake manifold temperature (e.g., surge tank temperature) may be stored with respect to the manifold temperature sensor 135, the exhaust temperature may be stored with respect to the exhaust temperature sensor 170, the upstream oxygen concentration (or the output voltage of the upstream oxygen sensor 175) may be stored with respect to the upstream oxygen sensor 175, and the downstream oxygen concentration (or the output voltage of the downstream oxygen sensor) may be stored with respect to the downstream oxygen sensor 185.

Subsequently at step S520, the ECU 200 determines whether a drop in the output torque Tq of the engine ENG is detected. When a misfire occurs, the output torque of the engine ENG is instantaneously reduced. In this case, the engine ENG does not rotate smoothly and vibration occurs, which is generally referred to as engine roughness occuring. That is, the step S20 may be understood to determine whether the engine roughness occurs.

When the output torque drop of the engine ENG is not detected (S520-No), the ECU 200 proceeds to the initial step S510.

When the output torque drop of the engine ENG is detected (S520-Yes), the ECU 200 controls the engine ENG by an optimal air/fuel ratio at step S525, and counts the occurrence of the output torque drop at step S530.

The step S525 of controlling the engine ENG by the optimal air/fuel ratio is to improve accuracy of the diagnosis based on the detection value of the upstream oxygen sensor 175. That is, depending on a driver's operation (e.g., foot on a brake pedal, an accelerator pedal, and the like), the ECU 200 may control the engine ENG by an air/fuel ratio (e.g., an air/fuel ratio for maximum output torque or for best fuel efficiency) that is different from the optimal air/fuel ratio (e.g., a theoretical air/fuel ratio). It may be understood that, in this case, the upstream oxygen sensor 175 may output the oxygen concentration value slightly different from 0 even in the normal combustion. That is, at the step S525, the engine ENG is controlled at a driving state that outputs zero (0) value of the oxygen concentration at the upstream oxygen sensor 175.

At the step S530, the number of the occurrence of the output torque drop in the combustion of the engine ENG in such a control state is counted, which is hereinafter referred to as output torque drop count.

Subsequently at step S535, the ECU 200 determines whether the output torque drop count of the engine ENG in the optimal air/fuel ratio mode is above a predetermined number.

When the output torque drop count of the engine ENG in the optimal air/fuel ratio mode is below the predetermined number (S535-No), the ECU 200 proceeds to the initial step S510. At this time, the ECU 200 may reset an accumulated output torque drop count.

When the output torque drop count of the engine ENG in the optimal air/fuel ratio mode is above the predetermined number (S535-Yes), the ECU 200 determines, at step S540, whether a misfire code for an injection fail and/or an ignition fail is already stored in the memory.

That is, the ECU 200 is basically capable of determining the misfire due to an injection fail and/or an ignition fail based on the detection values from the sensing unit 100, and when the misfire due to an injection fail and/or an ignition fail is determined, the ECU 200 is configured to store a corresponding misfire code for the injection/ignition fail in the memory 210.

When the misfire code for an injection fail and/or an ignition fail is already stored in the memory 210 (S540-Yes), the ECU 200 additionally stores, at step S545, a further code indicating that a misfire has additionally occurred in the memory 210.

When the misfire code for an injection fail and/or an ignition fail is already stored in the memory 210, the ECU 200 may possibly be performing a corresponding fail-safe control, such as a limp-home mode. Therefore, at the step S545, the ECU 200 does not further determine whether the detected misfire is due to exhaust valve leakage, and merely stores the code indicating that another misfire has additionally occurred.

At the step S545, the ECU 200 may also store a code indicating that it is not further determined whether the misfire is due to exhaust valve leakage. Such stored data allows for a more accurate judgment in the maintenance of the vehicle by allowing misfire records to be reviewed later through OBD or the like.

When the misfire code for an injection fail and/or an ignition fail is not stored in the memory 210 (S540-No), the ECU 200 determines, at step S550, whether a misfire due to exhaust valve leakage has occurred.

Whether the misfire due to exhaust valve leakage has occurred may be determined based on a combination of at least one condition of:

(a first condition) whether the intake air amount detected by the MAF sensor 120 has decreased compared to the normal combustion,

(a second condition) whether an upstream oxygen concentration detected by the upstream oxygen sensor 175 has increased compared to the normal combustion,

(a third condition) whether the intake manifold pressure detected by the MAP sensor 130 has increased compared to the normal combustion,

(a fourth condition) whether exhaust temperature detected by the exhaust temperature sensor 170 has increased compared to the normal combustion, and

(a fifth condition) whether intake manifold temperature detected by the manifold temperature sensor 135 has increased compared to the normal combustion,

For example, the ECU 200 may determine that the misfire due to exhaust valve leakage when the first condition, the second condition, the third condition, the fourth condition, and the fifth condition are all satisfied, Meanwhile, as another example, excluding the fourth condition, the first condition, the second condition, the third condition, and the fifth condition are all satisfied, the ECU 200 may determine that the misfire due to exhaust valve leakage.

FIG. 2 illustrates that, a system for diagnosing a misfire of an engine according to an exemplary embodiment, the sensing unit 100 includes, for example, the MAF sensor 120, the MAP sensor 130, the manifold temperature sensor 135, the exhaust temperature sensor 170, and the upstream oxygen sensor 175.

However, it has been described above that the sensing unit 100 may exclude one or more sensors, for example, the manifold temperature sensor 135.

In this case, whether the misfire due to exhaust valve leakage has occurred may be determined based on a combination of at least one condition of:

(the first condition) whether the intake air amount detected by the MAF sensor 120 has decreased compared to the normal combustion,

(the second condition) whether the upstream oxygen concentration detected by the upstream oxygen sensor 175 has increased compared to the normal combustion,

(the third condition) whether the intake manifold pressure detected by the MAP sensor 130 has increased compared to the normal combustion, and

(the fourth condition) whether exhaust temperature detected by the exhaust temperature sensor 170 has increased compared to the normal combustion,

excluding the fifth condition,

For example, the ECU 200 may determine that the misfire due to exhaust valve leakage when the first condition, the second condition, the third condition, and the fourth condition are all satisfied. Meanwhile, as another example, excluding the fourth condition, the first condition, the second condition, and the third condition are all satisfied, the ECU 200 may determine that the misfire due to exhaust valve leakage.

When the misfire due to exhaust valve leakage has not occurred (S550-No), the ECU 200 proceeds to the initial step S510. At this time, the ECU 200 may reset the accumulated output torque drop count.

When the misfire due to exhaust valve leakage has occurred (5550-Yes), the ECU 200 generates, at step S555, a misfire code corresponding to the misfire due to exhaust valve leakage, and stores the failure code in the memory 210. At the step S555, the ECU 200 may also store a further information in the memory 210, such as a vehicle running state (e.g., engine speed and the like), the driver's operation (e.g., throttle input and the like), and engine control state (e.g., a fuel injection amount, an ignition timing, and an actual throttle opening).

Subsequently at step S560, the ECU 200 may warn the driver to check the engine by lighting the engine warning lamp 290.

In addition, when the misfire due to exhaust valve leakage has occurred (S550-Yes), the ECU 200 controls the engine ENG, at step S565, by avoiding the engine control state in which the misfire due to exhaust valve leakage has occurred.

In more detail, while the ECU 200 controls the engine ENG according to the driver's operation such as a throttle input and the vehicle running state such as an engine speed, the same operation of the driver may be input in the same vehicle running state. At this time, the ECU 200 is expected to have the misfire due to exhaust valve leakage when the same control (i.e., the same fuel injection and timing, the same ignition timing, and the same throttle opening) is applied to the engine ENG. Therefore, the ECU 200 may control the engine ENG by avoiding the driving point (e.g., the engine speed, the fuel injection amount and timing, the ignition timing, the throttle opening, and the like) where the misfire due to exhaust valve leakage has occurred.

For example, when a current driving point corresponds to the driving point where the misfire due to exhaust valve leakage has occurred, the ECU 200 may retard the ignition timing of the spark plug 155 through the ignition coil 160 to avoid the driving point of the misfire due to exhaust valve leakage, thereby preventing the same misfire.

As such, since the ECU 200 may control the engine ENG by avoiding the driving point where the misfire due to exhaust valve leakage has occurred, engine damage due to abnormal combustion may be minimized, and output power loss may be minimized while the driver is moving the vehicle for maintenance.

While this disclosure has been described in connection with what is presently considered to be practical exemplary embodiments, it is to be understood that the disclosure is not limited to the disclosed embodiments. On the contrary, it is intended to cover various modifications and equivalent arrangements included within the spirit and scope of the appended claims.

What is claimed is:

1. A system for diagnosing a misfire of an engine, comprising:
a sensing unit including at least one sensor for detecting at least one detection value associated with an operation of the engine; and
an electronic control unit configured to determine whether a misfire of the engine due to exhaust valve leakage has occurred based on the detection values from the sensing unit, and perform an operation corresponding to the misfire due to exhaust valve leakage when the misfire due to exhaust valve leakage has occurred,
wherein the electronic control unit stores a misfire code for exhaust valve leakage in a memory when the misfire due to exhaust valve leakage has occurred.

2. The system of claim 1, wherein the electronic control unit is configured to:
detect an output torque drop of the engine;
control the engine by an optimal air/fuel ratio when the output torque drop of the engine is detected;
count an output torque drop of the engine while controlling the engine by the optimal air/fuel ratio; and
determine whether the misfire due to exhaust valve leakage has occurred when the output torque drop count is above a predetermined number.

3. The system of claim 2, wherein the misfire code for exhaust valve leakage comprises information on a misfire occurrence driving point where the misfire due to exhaust valve leakage has occurred.

4. The system of claim 3, wherein, when the misfire due to exhaust valve leakage has occurred, the electronic control unit controls the engine at a driving point avoiding the misfire occurrence driving point.

5. The system of claim 2, wherein the electronic control unit is configured to:
determine whether a code corresponding to a misfire due to an injection fail and/or an ignition fail is already stored in the memory when the output torque drop count is above the predetermined number;
additionally store a further code indicating that a misfire has additionally occurred when the code corresponding to a misfire due to an injection fail and/or an ignition fail is already stored in the memory; and
perform the determining of whether the misfire due to exhaust valve leakage has occurred only when the code corresponding to a misfire due to an injection fail and/or an ignition fail is not stored in the memory.

6. The system of claim 1, wherein the sensing unit comprises:
a MAF sensor for detecting an intake air amount supplied to the engine;
an upstream oxygen sensor for detecting upstream oxygen concentration of a catalytic converter of the engine;
a MAP sensor for detecting an intake manifold pressure of the engine:
an exhaust temperature sensor for detecting an exhaust temperature of the engine; and
a manifold temperature sensor for detecting an intake manifold temperature the engine,
wherein the electronic control unit determines whether the misfire due to exhaust valve leakage has occurred based on a combination of at least one condition of:
(a first condition) whether the intake air amount detected by the MAF sensor has decreased compared to a normal combustion;
(a second condition) whether the upstream oxygen concentration detected by the upstream oxygen sensor has increased compared to the normal combustion;
(a third condition) the intake manifold pressure detected by the MAP sensor has increased compared to the normal combustion;
(a fourth condition) whether the exhaust temperature detected by the exhaust temperature sensor has increased compared to the normal combustion; and
(a fifth condition) whether the intake manifold temperature detected by the manifold temperature sensor has increased compared to the normal combustion.

7. The system of claim 6, wherein the electronic control unit determines that the misfire due to exhaust valve leakage has occurred when the first condition, the second condition, the third condition, and the fifth condition are all satisfied.

8. The system of claim 6, wherein the electronic control unit determines that the misfire due to exhaust valve leakage has occurred when the first condition, the second condition, the third condition, the fourth condition, and the fifth condition are all satisfied.

9. The system of claim 1, wherein the sensing unit comprises:
a MAF sensor for detecting an intake air amount supplied to the engine,
an upstream oxygen sensor for detecting upstream oxygen concentration of a catalytic converter of the engine,
a MAP sensor for detecting an intake manifold pressure of the engine, and
an exhaust temperature sensor for detecting an exhaust temperature of the engine,
wherein the electronic control unit determines whether the misfire due to exhaust valve leakage has occurred, based on a combination of at least one condition of:
(a first condition) whether the intake air amount detected by the MAF sensor has decreased compared to a normal combustion;
(a second condition) whether the upstream oxygen concentration detected by the upstream oxygen sensor has increased compared to the normal combustion;
(a third condition) the intake manifold pressure detected by the MAP sensor has increased compared to the normal combustion; and
(a fourth condition) whether the exhaust temperature detected by the exhaust temperature sensor has increased compared to the normal combustion.

10. The system of claim 9, wherein the electronic control unit determines that the misfire due to exhaust valve leakage has occurred when the first condition, the second condition, and the third condition are all satisfied.

11. The system of claim 9, wherein the electronic control unit determines that the misfire due to exhaust valve leakage has occurred when the first condition, the second condition, the third condition, and the fourth condition are all satisfied.

12. A method for diagnosing a misfire of an engine, comprising:
receiving a detection value from a sensing unit including at least one sensor for detecting at least one detection value associated with an operation of the engine;
detecting an output torque drop of the engine;
controlling the engine by an optimal airifuel ratio when the output torque drop of the engine is detected;
counting an output torque drop of the engine while controlling the engine by the optimal airlfuel ratio;
determining whether a misfire due to exhaust valve leakage has occurred when the output torque drop count is above a predetermined number; and
storing a misfire code in a memory when the misfire due to exhaust valve leakage has occurred.

13. The method of claim 12. wherein the storing of the misfire code into the memory comprises storing information on a misfire occurrence driving point where the misfire due to exhaust valve leakage has occurred in the memory.

14. The method of claim 13, further comprising controlling the engine at a driving point avoiding the misfire occurrence driving point when the misfire due to exhaust valve leakage has occurred.

15. The method of claim 12, further comprising:
determining whether a code corresponding to a misfire due to an injection fail and/or an ignition fail is already stored in the memory when the output torque drop count is above a predetermined number; and
additionally storing a further code indicating that a misfire has additionally occurred when the code corresponding to a misfire due to an injection fail and/or an ignition fail is already stored in the memory,
wherein the determining of whether the misfire due to exhaust valve leakage has occurred is performed only when the code corresponding to a misfire due to an injection fail and/or an ignition fail is not stored in the memory.

16. The method of claim 12, wherein whether the misfire due to exhaust valve leakage has occurred is determined based on a combination of at least one condition of:
(a first condition) whether an intake air amount supplied to the engine has decreased compared to a normal combustion;
(a second condition) whether an upstream oxygen concentration of a catalytic converter of the engine has increased compared to the normal combustion;
(a third condition) whether an intake manifold pressure of the engine has increased compared to the normal combustion;
(a fourth condition) whether an exhaust temperature of the engine has increased compared to the normal combustion; and
(a fifth condition) whether an intake manifold temperature of the engine has increased compared to the normal combustion.

17. The method of claim 16, wherein it is determined that the misfire due to exhaust valve leakage has occurred when the first condition, the second condition, the third condition, and the fifth condition are all satisfied.

18. The method of claim 16, wherein it is determined that the misfire due to exhaust valve leakage has occurred when the first condition, the second condition, the third condition, the fourth condition, and the fifth condition are all satisfied, a method for diagnosing a misfire of an engine.

19. The method of claim 12, wherein whether the misfire due to exhaust valve leakage has occurred is determined based on a combination of at least one condition of:
(a first condition) whether an intake air amount supplied to the engine has decreased compared to a normal combustion;
(a second condition) whether an upstream oxygen concentration of a catalytic converter of the engine has increased compared to the normal combustion;
(a third condition) whether an intake manifold pressure of the engine has increased compared to the normal combustion; and
(a fourth condition) whether an exhaust temperature of the engine has increased compared to the normal combustion.

20. The method of claim 19, wherein it is determined that the misfire due to exhaust valve leakage has occurred when the first condition, the second condition, and the third condition are all satisfied.
